(19) United States
(12) Patent Application Publication
(10) Pub. No.: US 2025/0255502 A1
(43) Pub. Date: Aug. 14, 2025

(54) BLOOD PRESSURE MEASUREMENT DEVICE

(71) Applicant: OMRON HEALTHCARE Co., Ltd., Kyoto (JP)

(72) Inventors: Takaaki OKANISHI, Kyoto (JP); Yoshihide TOKKO, Kyoto (JP); Misaki TERUYAMA, Kyoto (JP)

(21) Appl. No.: 19/058,827

(22) Filed: Feb. 20, 2025

Related U.S. Application Data
(63) Continuation of application No. PCT/JP2023/038731, filed on Oct. 26, 2023.

(30) Foreign Application Priority Data
Feb. 1, 2023 (JP) 2023-014296

(51) Int. Cl. A61B 5/0235; A61B 5/00; A61B 5/021

(52) U.S. Cl. CPC A61B 5/0235; A61B 5/02141; A61B 5/681; A61B 2562/0247

(57) ABSTRACT

A blood pressure measurement device includes a case, a plurality of components including a pump provided in the case, a valve provided in the case, and a pressure sensor provided in the case, and a flow path plate unit in which a plurality of internal flow paths are formed, first ends of the plurality of internal flow paths being respectively connected to the plurality of components and second ends of the plurality of the internal flow paths being selectively connected to at least one of a plurality of cuffs and a plurality of testers configured to respectively test the plurality of components.

### CROSS-REFERENCE TO RELATED APPLICATIONS

This application is the U.S. national stage application filed pursuant to 35 U.S.C. 365 (c) and 120 as a continuation of International Patent Application No. PCT/JP2023/038731, filed Oct. 26, 2023, which application claims priority to Japanese Patent Application No. 2023-014296, filed Feb. 1, 2023, which applications are incorporated herein by reference in their entireties.

### TECHNICAL FIELD

The present invention relates to a blood pressure measurement device.

### BACKGROUND ART

In recent years, blood pressure measurement devices used for measuring blood pressure are being utilized as a means for checking a health state not only at a medical facility but also at home. A blood pressure measurement device detects vibration of the artery wall to measure blood pressure by, for example, inflating and contracting a cuff wound around the wrist or the like of a living body and detecting the pressure in the cuff by using a pressure sensor.

Further, as disclosed in JP 2022-116594 A, a wearable sphygmomanometer attached to the wrist is known as a blood pressure measurement device. Such a blood pressure measurement device uses a flow path plate unit for size reduction. Components such as a pressure sensor, an open-close valve, and a flow path resistor are connected to the flow path plate unit.

### CITATION LIST

### Patent Literature

Patent Document 1: JP 2022-116594 A

### SUMMARY OF INVENTION

### Technical Problem

In the blood pressure measurement device described above, after connecting each component, it is necessary to block unnecessary flow paths to test each component. However, when a mechanism that blocks unnecessary flow paths is provided in the flow path plate unit, the flow path plate unit increases in size, which poses the problem that testing cannot be performed without changing the size of the flow path plate unit.

It is therefore an object of the present invention to provide a blood pressure measurement device that enables individual testing of components provided in a flow path plate unit without an increase in size of the flow path plate unit.

### Solution to Problem

According to an aspect, a blood pressure measurement device includes a case, a plurality of components including a pump provided in the case, a valve provided in the case, and a pressure sensor provided in the case, and a flow path plate unit in which a plurality of internal flow paths are formed, first ends of the plurality of internal flow paths being respectively connected to the plurality of components and second ends of the plurality of internal flow paths being selectively connected to at least one of a plurality of cuffs and a plurality of testers configured to respectively test the plurality of components.

According to this aspect, each of the plurality of internal flow paths of the flow path plate unit can connect any of the components and one of the cuffs or the testers. Accordingly, the internal flow paths connected to the respective components of the flow path plate unit are independent from each other. Thus, when the blood pressure measurement device is in a usable state, the flow path plate unit can connect the components and the cuffs. Further, the components and the testers can be connected when the blood pressure measurement device is in a test state at the time of manufacture or maintenance, and the components and the testers can be sealed when testing is not performed. Thus, the blood pressure measurement device enables individual testing of the components provided in the flow path plate unit without an increase in size of the flow path plate unit.

In the blood pressure measurement device according to the aspect described above, the plurality of cuffs include a pressing cuff and a sensing cuff, the flow path plate unit includes a first flow path resistor and a second flow path resistor, and the plurality of internal flow paths include an internal flow path connecting the pressing cuff and the first flow path resistor, an internal flow path connecting the first flow path resistor and the sensing cuff, an internal flow path connecting the sensing cuff and the second flow path resistor, and an internal flow path connecting the second flow path resistor and the atmosphere.

According to this aspect, the blood pressure measurement device includes the internal flow paths capable of connecting the pressing cuff and the first flow path resistor, the first flow path resistor and the sensing cuff, the sensing cuff and the second flow path resistor, and the second flow path resistor and the atmosphere, making it possible to test the first flow path resistor and the second flow path resistor by connecting the testers to the internal flow paths connected to the first flow path resistor and the second flow path resistor.

In the blood pressure measurement device according to the aspect described above, the internal flow path connecting the pressing cuff and the first flow path resistor is further connected to the pump, and the internal flow path connecting the first flow path resistor and the sensing cuff and the internal flow path connecting the sensing cuff and the second flow path resistor are continuously and integrally formed.

According to this aspect, the blood pressure measurement device facilitates connection of a tester in place of the pressing cuff to the internal flow path connecting the pump, the pressing cuff, and the first flow path resistor, and connection of a tester in place of the sensing cuff to the internal flow path connecting the first flow path resistor, the second flow path resistor, and the sensing cuff. Thus, the blood pressure measurement device is capable of testing the first flow path resistor and the second flow path resistor.

In the blood pressure measurement device according to the aspect described above, the flow path plate unit includes a plurality of ports respectively connectable to the plurality of cuffs and the plurality of testers, and respectively continuous with the plurality of internal flow paths.

According to this aspect, the blood pressure measurement device enables selective connection of each cuff and each tester to the flow path plate unit via the plurality of ports. Thus, the blood pressure measurement device facilitates the attachment and detachment of the cuffs and the testers and the sealing of the ports, making it possible to easily test the various components.

### Advantageous Effects of Invention

According to the present invention, it is possible to provide a blood pressure measurement device that enables individual testing of components provided in a flow path plate unit without an increase in size of the flow path plate unit.

### BRIEF DESCRIPTION OF DRAWINGS

FIG. 1 is a perspective view illustrating a configuration of a blood pressure measurement device according to an embodiment of the present invention.

FIG. 2 is a side view illustrating the configuration of the blood pressure measurement device.

FIG. 3 is a side view illustrating the configuration of the blood pressure measurement device in a state of being attached to a wrist.

FIG. 4 is a block diagram illustrating the configuration of the blood pressure measurement device.

FIG. 5 is a cross-sectional view illustrating a configuration of a main portion of the blood pressure measurement device.

FIG. 6 is a plan view illustrating the configuration of the main portion of the blood pressure measurement device from a rear surface side of a device body.

FIG. 7 is a plan view illustrating the configuration of the main portion of the blood pressure measurement device with some configurations omitted from the rear surface side of the device body.

FIG. 8 is an exploded perspective view illustrating a configuration of the device body.

FIG. 9 is an exploded perspective view illustrating the configuration of the device body.

FIG. 10 is a perspective view schematically illustrating an example of a flow path plate unit of the blood pressure measurement device.

FIG. 11 is a block diagram illustrating an example of a fluid circuit of the blood pressure measurement device.

FIG. 12 is a block diagram illustrating an example of components provided to the flow path plate unit of the blood pressure measurement device during testing.

FIG. 13 is a perspective view schematically illustrating an example of the flow path plate unit of the blood pressure measurement device according to another embodiment of the present invention.

FIG. 14 is a block diagram illustrating an example of the fluid circuit of the blood pressure measurement device.

FIG. 15 is a block diagram illustrating an example of the components provided to the flow path plate unit of the blood pressure measurement device during testing.

FIG. 16 is a block diagram illustrating an example of the components provided to the flow path plate unit of the blood pressure measurement device during testing.

FIG. 17 is a perspective view schematically illustrating an example of the flow path plate unit of the blood pressure measurement device according to another embodiment of the present invention.

FIG. 18 is a block diagram illustrating an example of the fluid circuit of the blood pressure measurement device.

FIG. 19 is a block diagram illustrating an example of the components provided to the flow path plate unit of the blood pressure measurement device during testing.

### DESCRIPTION OF EMBODIMENTS

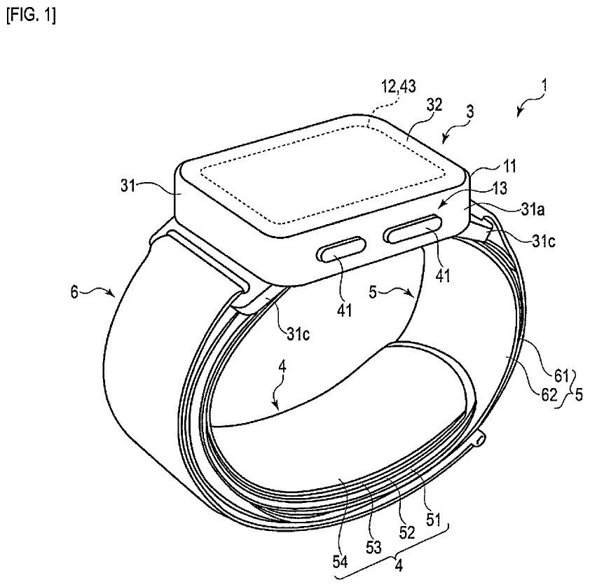

An example of a blood pressure measurement device 1 according to an embodiment of the present invention will be described below with reference to FIG. 1 to FIG. 12.

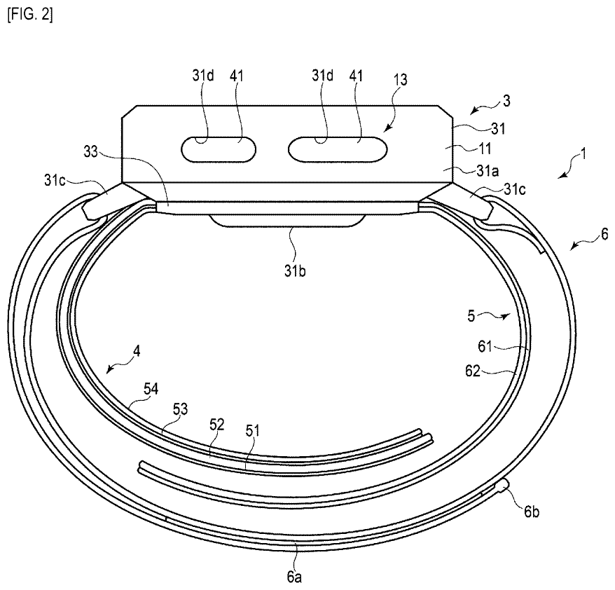

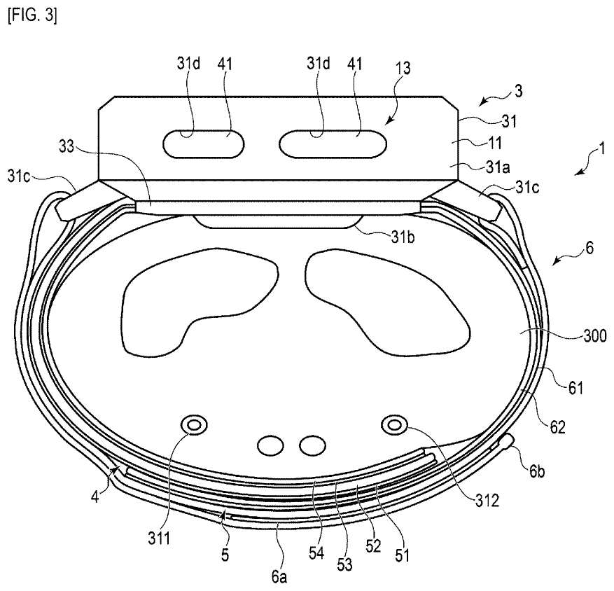

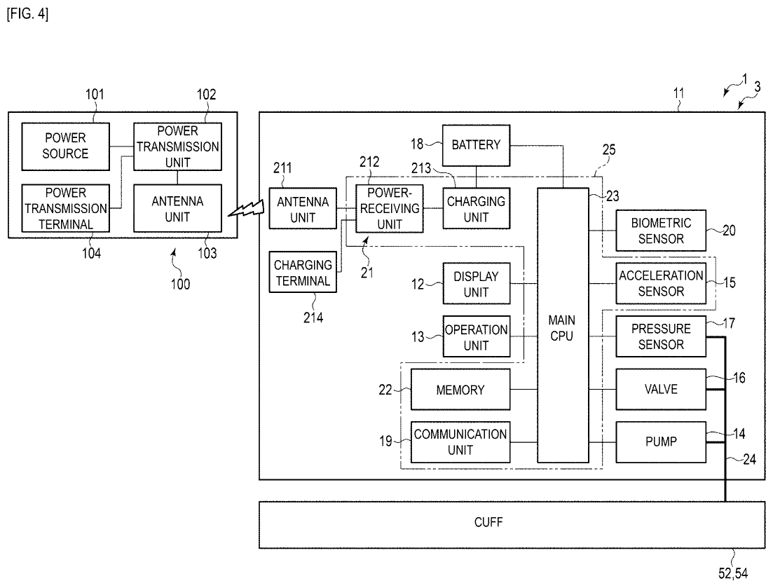

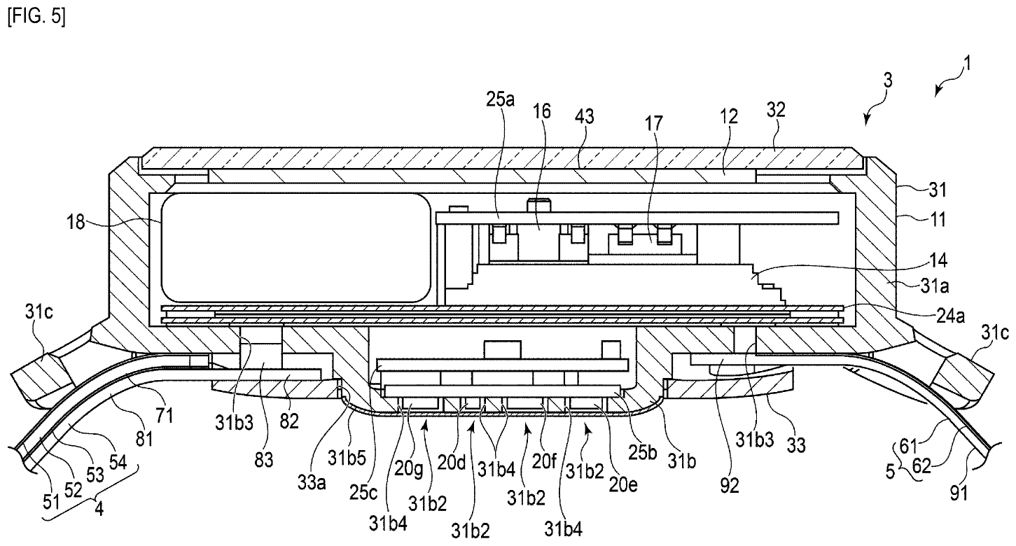

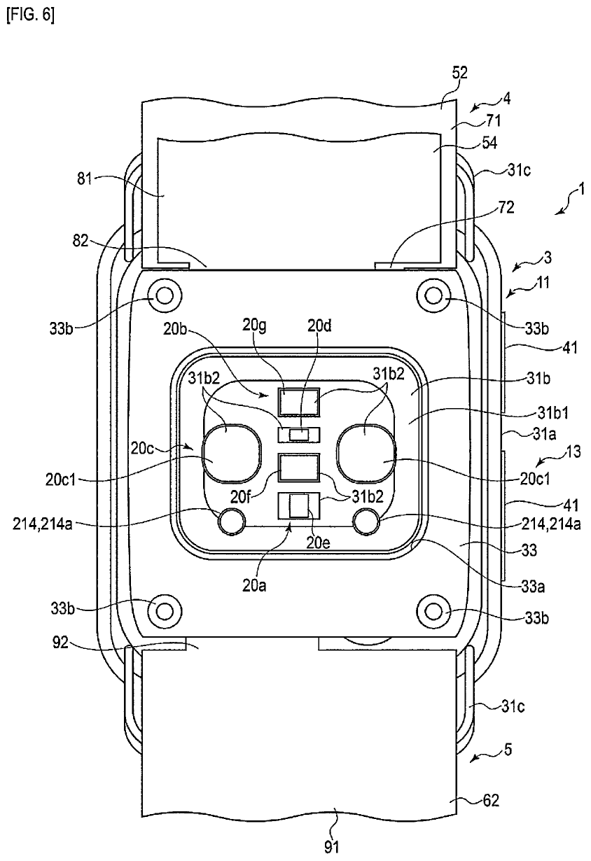

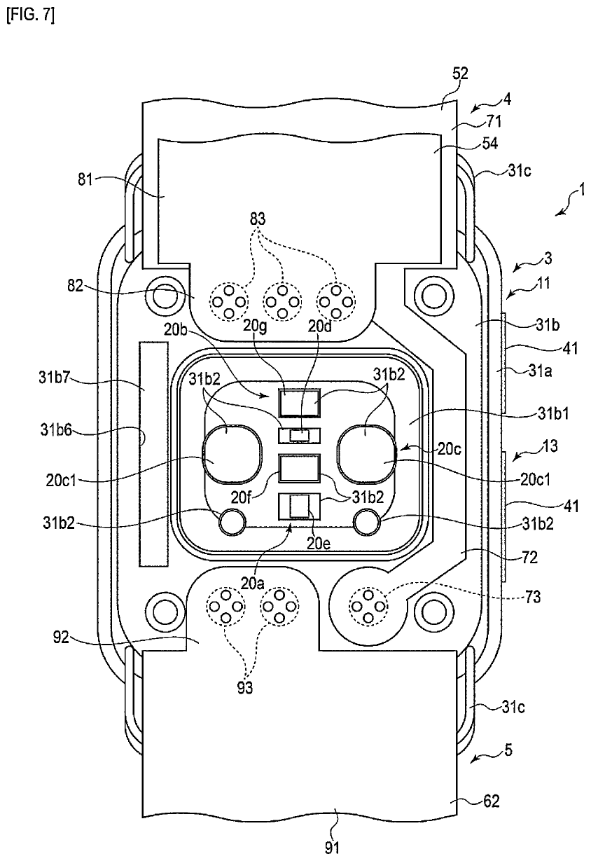

FIG. 1 is a perspective view illustrating a configuration of the blood pressure measurement device 1 according to the embodiment of the present invention. FIG. 2 is a side view illustrating the configuration of the blood pressure measurement device 1, and FIG. 3 is a side view illustrating the configuration of the blood pressure measurement device 1 in a state of being attached to a wrist 300. FIG. 4 is a block diagram illustrating the configuration of the blood pressure measurement device 1. FIG. 5 is a cross-sectional view illustrating a configuration of a device body 3, a portion of a first cuff structure 4, and a portion of a second cuff structure 5 of the blood pressure measurement device 1. FIG. 6 is a plan view illustrating the configuration of the device body 3, a portion of the first cuff structure 4, and a portion of the second cuff structure 5 of the blood pressure measurement device 1, and FIG. 7 is a plan view illustrating the configuration of the device body 3, a portion of the first cuff structure 4, and a portion of the second cuff structure 5 of the blood pressure measurement device 1 with a cuff cover 33 omitted.

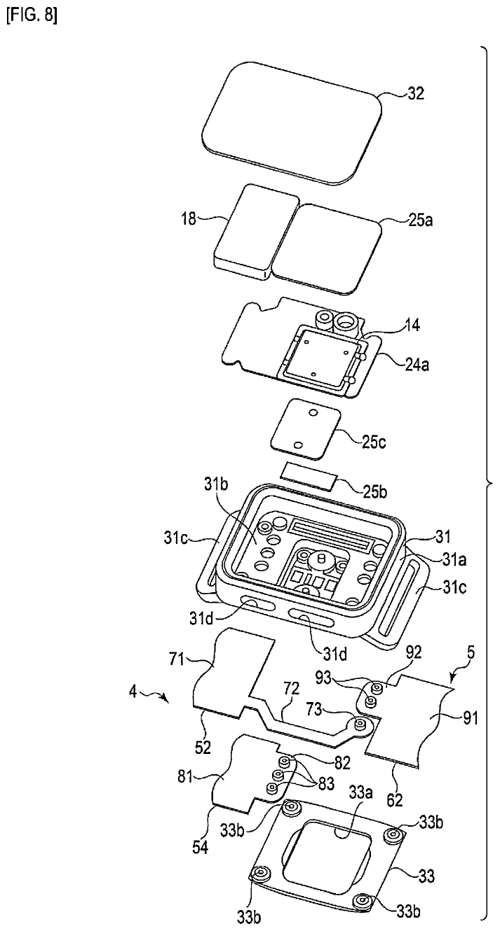

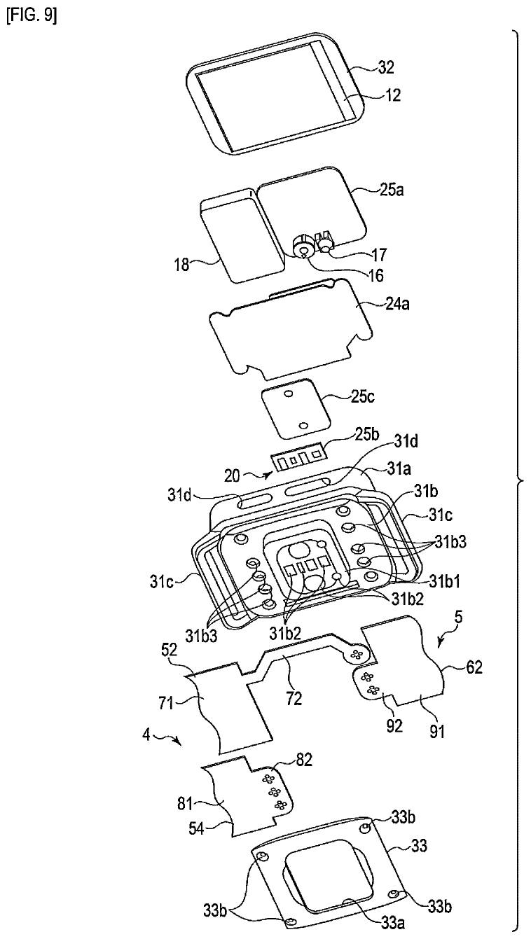

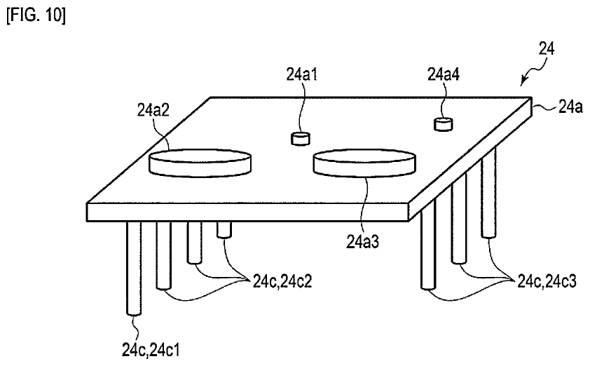

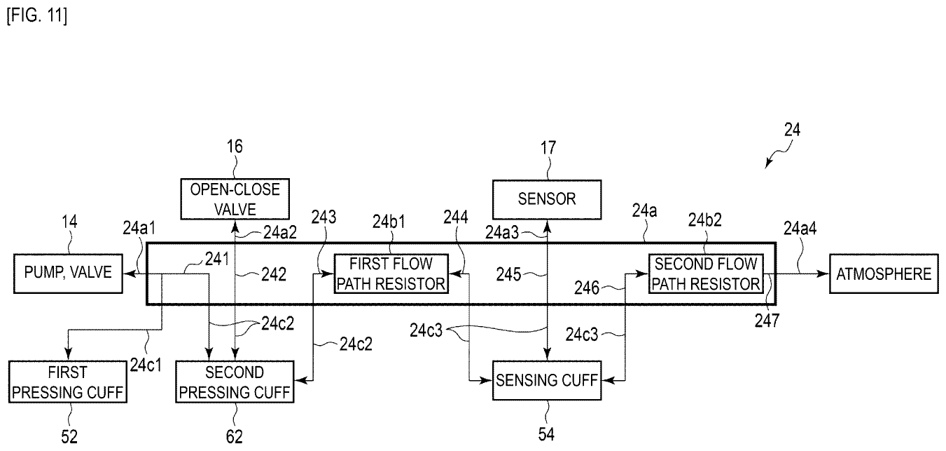

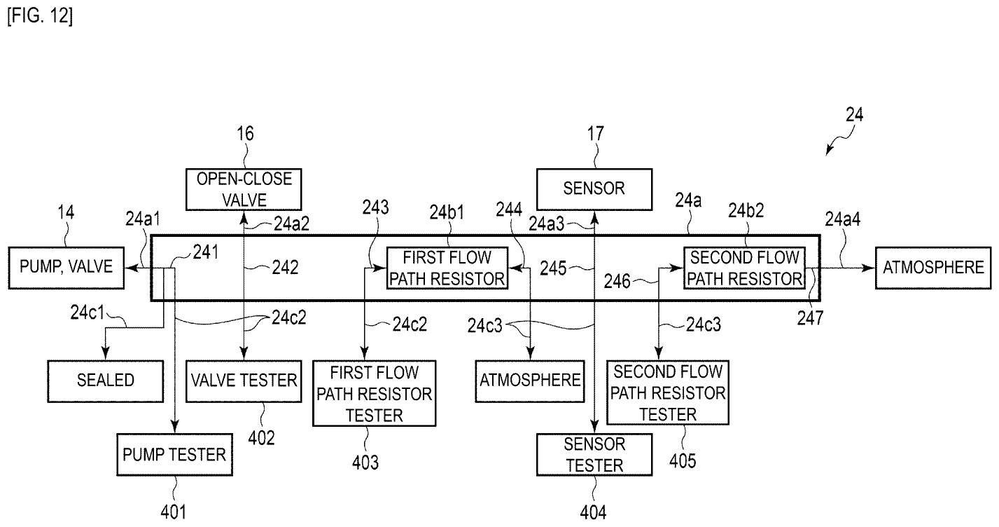

FIG. 8 is an exploded perspective view illustrating the configuration of the device body 3 from an upper surface side, and FIG. 9 is an exploded perspective view illustrating the configuration of the device body 3 from a lower surface side. FIG. 10 is a perspective view schematically illustrating an example of a flow path plate unit 24 of the blood pressure measurement device 1, FIG. 11 is a block diagram illustrating an example of the fluid circuit 24 of the blood pressure measurement device 1, and FIG. 12 is a block diagram illustrating an example of components provided to the flow path plate unit 24 of the blood pressure measurement device 1 during testing.

As illustrated in FIG. 1 to FIG. 3, the blood pressure measurement device 1 includes, for example, the device body 3, the first cuff structure 4, the second cuff structure 5, and a band 6. The blood pressure measurement device 1 is configured such that the first cuff structure 4 and the second cuff structure 5 extend from the device body 3 in opposite directions of the device body 3, with the band 6 covering the first cuff structure 4 and the second cuff structure 5, and is formed fixable to the wrist 300 that is a living body by the band 6.

As illustrated in FIG. 1 to FIG. 5, FIG. 8, and FIG. 9, the device body 3 includes, for example, a case 11, a display unit 12, an operation unit 13, a pump 14, an acceleration sensor 15, a valve 16, a pressure sensor 17, a battery 18, a communication unit 19, biometric sensors 20, a charging circuit unit 21, a memory 22, a processor 23, the fluid circuit 24, and a substrate 25.

The case 11 is a case that accommodates components of the device body 3. The case 11 accommodates, for example, the display unit 12, the operation unit 13, the pump 14, the acceleration sensor 15, the valve 16, the pressure sensor 17, the battery 18, the communication unit 19, the biometric sensors 20, the charging circuit unit 21, the memory 22, the processor 23, the fluid circuit 24, and the substrate 25.

As illustrated in FIG. 5 to FIG. 7, the case 11 includes, for example, an outer case 31, a windshield 32 that covers an upper opening of the outer case 31, and the cuff cover 33 provided below the outer case 31.

The outer case 31 is formed in a bottomed tubular shape such as, for example, a cylindrical, rectangular tubular, or polygonal tubular shape with a bottom portion. In the present embodiment, an example is illustrated in which the outer case 31 is formed in a bottomed rectangular tubular shape. As a specific example, the outer case 31 includes a peripheral wall portion 31a having a rectangular tubular shape, a bottom portion 31b provided at the peripheral wall portion 31a, and a pair of loop portions 31c provided at a pair of surfaces among four outer peripheral surfaces of the peripheral wall portion 31a.

In the peripheral wall portion 31a, for example, an opening 31d in which part of the operation unit 13 is disposed is formed. For example, the opening 31d is formed in one surface of the peripheral wall portion 31a that is different from the two surfaces provided with the pair of loop portions 31c. Further, for example, two of the openings 31d are formed in one surface of the peripheral wall portion 31a.

The bottom portion 31b constitutes a rear surface (bottom portion 31b) of the case 11 (outer case 31). The bottom portion 31b, for example, partially projects, facilitating contact with the wrist 300. For example, the bottom portion 31b includes a projection portion 31b1 formed by a center side of an outer surface side being projected in a rectangular shape and then a center side of an inner surface side of the bottom portion 31b being recessed in a rectangular shape. Further, in the bottom portion 31b are formed a plurality of window portions 31b2 in which the biometric sensors 20 are disposed and a plurality of hole portions 31b3 in which connection portions 73, 83 described below are disposed as members that fluidly connect the first cuff structure 4 and the second cuff structure 5 to the pump 14.

The biometric sensors 20 are disposed on the inner surface side of the projection portion 31b1. The projection portion 31b1 constitutes a sensor unit including at least one of the biometric sensors 20 or a charging terminal 214 disposed on the rear surface of the case 11. Here, the biometric sensors 20 and the charging terminal 214 being disposed on the rear surface of the case 11 means that some of the components constituting the biometric sensors 20 are exposed on the rear surface of the case 11 or are disposed on the rear surface of the case 11 with a member (for example, a cover 31b5 described below) other than the first cuff structure 4 and the second cuff structure 5 interposed therebetween.

The window portions 31b2 are formed in the projection portion 31b1. For example, as illustrated in FIG. 5, the window portions 31b2 are formed by a plurality of openings 31b4 formed at a plurality of locations of the projection portion 31b1 and by the cover 31b5 having transparency, made of glass, a resin material, or the like, and covering these openings 31b4. The cover 31b5 covers, for example, an outer surface of the projection portion 31b1.

The plurality of hole portions 31b3 are respectively formed between the projection portion 31b1 and each loop portion 31c in the bottom portion 31b. In the present embodiment, the plurality of hole portions 31b3 are formed between the projection portion 31b1 and one loop portion 31c, and then a plurality of hole portions 31b3 are formed between the projection portion 31b1 and the other loop portion 31c. The plurality of hole portions 31b3 provided adjacent to each loop portion 31c are aligned in one direction such as, for example, a direction orthogonal to an opposing direction of the pair of loop portions 31c. That is, in the bottom portion 31b which is the rear surface of the case 11, the plurality of hole portions 31b3 to which a portion of the configuration of the first cuff structure 4 is fixed at one end portion in the one direction are disposed at positions away from the projection portion 31b1, and then the plurality of hole portions 31b3 to which another portion of the configuration of the first cuff structure 4 as well as the second cuff structure 5 are fixed at the other end portion in the one direction are disposed at positions away from the projection portion 31b1 toward the one direction. Note that the number and the arrangement of the hole portions 31b3 are set as appropriate in accordance with a number and an arrangement of a plurality of ports 24c described below formed in a flow path plate 24a.

Further, a vent 31b6 having a slit shape long in the one direction is formed in the bottom portion 31b, and the bottom portion 31b includes a waterproof moisture-permeable sheet 31b7 covering this vent 31b6. The waterproof moisture-permeable sheet 31b7 is formed so as to prevent moisture from entering the case 11 from the outside through the vent 31b6 of the bottom portion 31b and to facilitate ventilation between the inside and the outside of the case 11. Here, the vent 31b6 and the waterproof moisture-permeable sheet 31b7 are provided at positions not overlapping the first cuff structure 4 and the second cuff structure 5 fixed to the bottom portion 31b, that is, at positions separated from the first cuff structure 4 and the second cuff structure 5. For example, in the direction orthogonal to the opposing direction of the pair of loop portions 31c, a flow path body 72 of a first pressing cuff 52 described below of the first cuff structure 4 is provided adjacent to one side of the projection portion 31b1, and the vent 31b6 and the waterproof moisture-permeable sheet 31b7 are provided adjacent to the other side of the projection portion 31b1.

The loop portion 31c is formed so that the band 6 can be passed therethrough and the band 6 can be fixed or the band 6 can be folded back. For example, the loop portion 31c is a member having a rectangular ring shape including an opening long in one direction through which the band 6 can be inserted. The loop portion 31c is integrally formed on an outer side surface of the outer case 31. One end of the band 6 is fixed to one loop portion 31c of the pair of loop portions 31c, and then the band 6 is folded back to the other loop portion 31c.

The windshield 32 is a glass plate having a shape similar to that of an outer peripheral edge of the outer case 31, and is a rectangular shape in the present embodiment. Note that the windshield 32 is not limited to a glass plate as long as the material has transparency or translucency.

The cuff cover 33 covers the bottom portion 31b of the outer case 31. In the cuff cover 33, the projection portion 31b1 of the bottom portion 31b is disposed and an opening 33a exposing the projection portion 31b1 to the outside is formed. For example, when the cuff cover 33 is fixed to the bottom portion 31b, the projection portion 31b1 is formed to a thickness and into a shape projecting from a main surface of the cuff cover 33 facing the wrist 300. Further, for example, the cuff cover 33 is formed with holes 33b in which screws are disposed at four corners, and is detachably fixed to the bottom portion 31b of the outer case 31 of the case 11 by screws or the like.

The cuff cover 33 covers end portions of the first cuff structure 4 and the second cuff structure 5 disposed on the bottom portion 31b, and thus the end portions of the first cuff structure 4 and the second cuff structure 5 are disposed in gaps that occur with the bottom surface 31b and the first cuff structure 4 and the second cuff structure 5 are fixed to the bottom surface 31b. As a specific example, the cuff cover 33 covers part of the flow path body 72 and the connection portion 73 of the first pressing cuff 52, a flow path body 82 and the plurality of connection portions 83 of a sensing cuff 54, and a flow path body 92 and a plurality of connection portions 93 of a second pressing cuff 62 described below. Then, the cuff cover 33 fixes the first cuff structure 4 and the second cuff structure 5 by restricting movement of the covered portion of part of the first pressing cuff 52, the sensing cuff 54, and the second pressing cuff 62 in a direction away from the bottom portion 31b. Note that a configuration may be adopted in which the cuff cover 33 includes a buffer material between the first cuff structure 4 and the second cuff structure 5.

The display unit 12 is disposed directly below the windshield 32. The display unit 12 is electrically connected to the processor 23. The display unit 12 is, for example, a liquid crystal display or an organic electroluminescence display. As a specific example, the display unit 12 is an organic light-emitting diode (OLED). The display unit 12 displays various information including the date and time, measurement results of blood pressure values such as the systolic blood pressure and diastolic blood pressure, heart rate, and the like, and information such as the charge state and remaining charge of the battery 18. The display unit 12 is formed in, for example, the same shape as that of the windshield 32 or a shape slightly smaller than that of the windshield 32 in plan view.

The operation unit 13 is configured to enable input of a command from a user. The operation unit 13 includes, for example, a plurality of buttons 41 provided on the case 11, a sensor that detects operation of the buttons 41, and a touch panel 43 provided on the display unit 12 or the windshield 32. When operated by the user, the operation unit 13 converts a command into an electrical signal. The sensor and the touch panel 43 are electrically connected to the processor 23 and output electrical signals to the processor 23.

The pump 14 is, for example, a piezoelectric pump. The pump 14, for example, compresses air as a fluid and supplies the compressed air through the fluid circuit 24 to an air bag 71 of the first pressing cuff 52 described below and an air bag 81 of the sensing cuff 54 of the first cuff structure 4, and an air bag 91 of the second pressing cuff 62 described below of the second cuff structure 5. The pump 14 is electrically connected to the processor 23. The pump 14 includes therein a valve mechanism that opens and closes flow paths connected to the flow path plate 24a.

The acceleration sensor 15 is, for example, a 3-axis acceleration sensor. The acceleration sensor 15 measures acceleration and outputs an analog signal, for example. The acceleration sensor 15 is connected to the processor 23 via, for example, an A/D conversion circuit.

The valve 16 is, for example, an open-close valve. The valve 16 opens and closes a fluid circuit connecting the pump 14 and the first cuff structure 4 and/or the second cuff structure 5 and/or a fluid circuit connecting the first cuff structure 4 and the outside (atmosphere) in the fluid circuit 24. In the present embodiment, the valve 16 opens and closes a fluid circuit (a second internal flow path 242 described below) connected to the second pressing cuff 62 described below of the second cuff structure 5. The valve 16 is electrically connected to the processor 23. For example, the valve 16 is opened and closed by the control of the processor 23.

As a specific example, the valve 16 is a valve that releases air supplied to the first cuff structure 4 and the second cuff structure 5 to the atmosphere. When air is to be supplied to the first pressing cuff 52, the second pressing cuff 62, and the sensing cuff 54 during blood pressure measurement, for example, the valve 16 is controlled by the processor 23 so as to be switched to the closed state. Further, when the air in the first pressing cuff 52, the second pressing cuff 62, and the sensing cuff 54 is to be exhausted, the valve 16 is controlled by the processor 23 so as to be switched from the closed state to the open state. Further, the valve 16 may be formed so that the opening degree is adjustable.

The pressure sensor 17 is provided, for example, in the fluid circuit 24. The pressure sensor 17 detects the pressures of the first pressing cuff 52 and/or the sensing cuff 54. The pressure sensor 17 detects the pressure of the sensing cuff 54, for example. The pressure sensor 17 is electrically connected to the processor 23 via an A/D conversion circuit, for example, converts the detected pressure into an electrical signal, and outputs the electrical signal to the processor 23.

The battery 18 is, for example, a secondary battery such as a rechargeable lithium ion battery. The battery 18 is electrically connected to the processor 23. The battery 18 supplies power to the processor 23. The battery 18 supplies power for driving to the respective configurations of the processor 23 and to the display unit 12, the operation unit 13, the pump 14, the acceleration sensor 15, the valve 16, the pressure sensor 17, the communication unit 19, and the biometric sensors 20 via the processor 23.

The communication unit 19 is configured to be capable of transmitting and receiving information to and from an external device wirelessly and/or by wire. The communication unit 19 is, for example, a wireless communication module conforming to a wireless communication standard. The communication unit 19, for example, transmits information, such as information controlled by the processor 23 and measured blood pressure values and pulse, to an external device, and receives a program for software updates or the like from an external device and transmits the program and the like to a control unit. In the present embodiment, the external device is, for example, an external terminal such as a smartphone, a tablet terminal, a personal computer, or a smart watch.

In the present embodiment, the communication unit 19 and the external terminal may be directly connected, or may be connected over a network. The communication unit 19 and the external terminal may be connected via a mobile communication network, such as 4G or 5G, and a wireless communication line, such as WiMAX or Wi-Fi (trade names). Further, the communication unit 19 and the external device may be connected by a wireless communication means, such as Bluetooth Low Energy (BLE; trade name), near field communication (NFC), and infrared communication. Further, the communication unit 19 may include, for example, a general-purpose connector such as a micro-universal serial bus (USB) in addition to a wireless communication module, and a dedicated connector for the blood pressure measurement device 1, and may be connected to the external terminal directly or via a wired communication line such as a local area network (LAN) connection using various cables such as a USB cable. Thus, a configuration may be adopted in which the communication unit 19 includes a plurality of communication means, such as a wireless antenna and a micro-USB connector. Note that the connector for wired communication may be a dedicated connector for the blood pressure measurement device 1.

The biometric sensors 20 are sensors formed so as to be capable of detecting biological information by being in contact with or by facing the wrist 300. The biometric sensors 20 each convert the detected biological information into an electrical signal and output the electrical signal to the processor 23. The biometric sensor 20 may be, for example, a sensor that measures a physical quantity such as a heart rate or a body temperature, or may be a sensor that measures a chemical value such as a blood sugar level or a blood oxygen concentration. In the present embodiment, the biometric sensors 20 include, for example, a photoplethysmography (PPG) sensor 20a, a peripheral oxygen saturation (SpO2) sensor 20b, and an electrocardiogram (ECG) sensor 20c.

For example, the PPG sensor 20a measures the heart rate by photoplethysmography. For example, the PPG sensor 20a includes a first LED 20d, a second LED 20e, and a first photodiode (PD) 20f.

For example, the SpO2 sensor 20b measures a saturation (transcutaneous arterial oxygen saturation). The SpO2 sensor 20b includes the second LED 20e and a second PD 20g. Here, for example, the PPG sensor 20a and the SpO2 sensor 20b share the second LED 20e.

For example, the ECG sensor 20c measures a flow of electricity in the heart to acquire an electrocardiographic waveform. For example, the ECG sensor 20c includes a pair of electrocardiogram.

The charging circuit unit 21 includes, for example, an antenna unit 211, a power-receiving unit 212, a charging unit 213, and the charging terminal 214. The charging circuit unit 21 charges the battery 18 by wire power transfer and/or wireless power transfer. For example, the charging circuit unit 21 receives transmission power transmitted by the antenna unit 211 from an antenna unit 103 of a power transmission device 100 externally provided, and charges the battery 18. Further, for example, the charging circuit unit 21 receives transmission power transmitted by the charging terminal 214 from a power transmission terminal 104 of the power transmission device 100 externally provided, and charges the battery 18. That is, the charging circuit unit 21 charges the battery 18 by selectively using the antenna unit 211 and the charging terminal 214 and receiving power from the power transmission device 100. Note that the charging circuit unit 21 may be formed so as to be capable of both wire power transfer and wireless power transfer or may be formed so as to be capable of only one of wire power transfer or wireless power transfer.

The antenna unit 211 receives transmission power from the antenna unit 103 of the power transmission device 100. The antenna unit 211 is, for example, a receiver coil as a power-receiving resonance circuit. The antenna unit 211 supplies the received power to the power-receiving unit 212. A power-receiving surface of the antenna unit 211 is formed in a planar shape. The antenna unit 211 is disposed, for example, in the case 11. As a specific example, the antenna unit 211 is provided adjacent to the display unit 12 on a side opposite to the windshield 32 of the display unit 12 in the case 11. The antenna unit 211 includes, for example, a resonance capacitor and constitutes the power-receiving resonance circuit.

The power-receiving unit 212 rectifies power received by the antenna unit 211 or the charging terminal 214, and supplies the rectified power to the charging unit 213. As a specific example, the power-receiving unit 212 rectifies the received power supplied from the antenna unit 211 and converts the rectified power from alternating current (AC) to direct current (DC). For example, the power-receiving unit 212 includes a rectifying circuit and a control circuit, controls the operation of the rectifying circuit by the control circuit, and outputs rectified DC power to the charging unit 213.

The charging unit 213 supplies the electric power supplied from the power-receiving unit 212 to the battery 18 as electric power for charging. For example, the charging unit 213 converts the electric power supplied from the power-receiving unit 212 into a predetermined current value and a predetermined voltage value and supplies the converted electric power to the battery 18. Further, for example, the charging unit 213 may include a circuit that outputs the charge state of the battery 18 to the power-receiving unit 212 and/or the processor 23.

The charging terminal 214 includes, for example, a pair of terminals 214a, and receives transmission power from the power transmission terminal 104 of the power transmission device 100 via the pair of terminals 214a.

The memory 22 includes, for example, a random access memory (RAM) and a read only memory (ROM). The memory 22 stores various data. For example, the memory 22 pre-stores, in a changeable manner, for example, various program data such as programs and applications for controlling the overall blood pressure measurement device 1 and the pump 14, settings data for setting various functions of the blood pressure measurement device 1, calculation data for calculating blood pressure values from the pressure measured by the pressure sensor 17, calculation data for calculating biological information such as a heart rate, a saturation, and an electrocardiographic waveform from the information measured by the biometric sensors 20.

The processor 23 controls the operation of the overall blood pressure measurement device 1 and the operations of the pump 14 and the valve 16 on the basis of the programs stored in the memory 22 and executes a predetermined operation (function). Further, the processor 23 executes the predetermined calculation, analysis, processing, or the like according to the read program. The processor 23 is an arithmetic device such as a central processing unit (CPU). The processor 23 may include, for example, a sub-CPU in addition to a main CPU. Further, the processor 23 displays a state or a result of the executed various operations, calculations, analyses, processing, and the like on the display unit 12 by a program or an application.

The fluid circuit 24 fluidly connects at least two or more of the pump 14, the valve 16, the pressure sensor 17, the first cuff structure 4, and the second cuff structure 5 provided in the case 11. The fluid circuit 24 may include, for example, components such as the flow path plate 24a that forms a flow path for the fluid supplied from the pump 14 to the first cuff structure 4, one or more orifices 24b serving as flow path resistors that control a supply amount and a pressure of the fluid supplied to the first cuff structure 4 and/or the second cuff structure 5, and a check valve that controls a flow direction of the fluid. In the present embodiment, the fluid circuit 24 is a flow path plate unit including the flow path plate 24a and a plurality of the orifices 24b.

The flow path plate 24a is formed by joining a plurality of plates formed of a resin material or a metal material and sheets or double-sided tape disposed between the plates adjacent to each other and in which slits that form the flow path are formed. The plurality of orifices 24b are provided in the flow path plate 24a. Hereinafter, in the present embodiment, the orifice 24b connected between the first pressing cuff 52 and the sensing cuff 54 described below of the first cuff structure 4 will be described as a first flow path resistor 24b1, and the orifice 24b connected to a secondary side of the sensing cuff 54 will be described as a second flow path resistor 24b2.

The substrate 25 includes, for example, a control board 25a, a sensor main board 25b, and a sensor sub-board 25c.

On the control board 25a, for example, the acceleration sensor 15, the valve 16, the pressure sensor 17, the communication unit 19, the circuit configuration of the charging circuit unit 21, the memory 22, and the processor 23 are mounted.

On the sensor main board 25b, the first LED 20d, the second LED 20e, the first PD 20f, and the second PD 20g used for the PPG sensor 20a and the SpO2 sensor 20b are mounted.

On the sensor sub-board 25c, various circuits and electronic components for configuring the PPG sensor 20a, the SpO2 sensor 20b, and the ECG sensor 20c are mounted. For example, the control board 25a, the sensor main board 25b, and the sensor sub-board 25c are electrically connected to one another.

Hereinafter, a specific example of the first cuff structure 4 will be described with reference to FIG. 1 to FIG. 3 and FIG. 5. As illustrated in FIG. 1 to FIG. 3 and FIG. 5, the first cuff structure 4 includes a first curler 51, the first pressing cuff 52, a back plate 53, and the sensing cuff 54. The first cuff structure 4 includes, for example, the first curler 51, the first pressing cuff 52, the back plate 53, and the sensing cuff 54. The first cuff structure 4 is configured by sequentially layering the first curler 51, the first pressing cuff 52, the back plate 53, and the sensing cuff 54 toward the wrist 300. Note that a configuration may be adopted in which the first cuff structure 4 does not include the first curler 51 and/or the back plate 53.

The first curler 51 is, for example, fixed, at one end side, to the bottom portion 31b provided on the wrist 300 side of the case 11. The first curler 51 is formed in a band-like shape that curves in a shape following a circumferential direction of the wrist 300. The first curler 51 is constituted by a resin material. The first curler 51 is formed of a material having a low hardness appropriate to provide flexibility and shape retainability. Here, “flexibility” refers to deformation of the shape of the first curler 51 in a radial direction at the time of application of an external force from the band 6 to the first curler 51. “Shape retainability” refers to the ability of the first curler 51 to maintain a pre-imparted shape when no external force is applied to the first curler 51. That is, the first curler 51 is formed of a resin material having a hardness that does not undergo compression deformation or is substantially without compression deformation but allows elastic deformation such as bending deformation in which the shape, particularly a curvature of a curved portion, changes. Accordingly, the first curler 51 is formed elastically deformable so as to be bent and deformed by application of an external force and to increase or decrease the internal space in which the wrist 300 is disposed in accordance with the shape of the wrist to which the first curler 51 is attached. For example, the first curler 51 is formed of a thermoplastic polyurethane resin (hereinafter referred to as TPU) or a polypropylene resin.

Further, the first curler 51, on one end side, is fixed to the case 11. Further, the first curler 51 is formed to such a length so as to face at least one of two arteries 311, 312 when the blood pressure measurement device 1 is attached to the wrist 300 having the longest circumference among the users who are expected to attach the device. Preferably, the first curler 51 is formed to such a length so as to face the two arteries 311, 312 when the blood pressure measurement device 1 is attached to the wrist 300 having the longest circumference among the users who are expected to attach the device. Further, the first curler 51 is set to such a length that the other end of the first curler 51 does not come into contact with the device body 3 when the blood pressure measurement device 1 is attached to the wrist 300 having the shortest circumference among the users who are expected to attach the device. In the first curler 51, for example, a portion extending from the case 11 of the device body 3 is curved by a predetermined radius of curvature so as to follow shapes of a side of one wrist 300 of the left and right wrists 300 and a palm side of the wrist 300.

The first pressing cuff 52 is fixed to an inner peripheral surface of the first curler 51 by double-sided tape, an adhesive, thermal welding, or the like. The first pressing cuff 52 is fluidly connected to the pump 14 through the fluid circuit 24. The first pressing cuff 52, at one main surface, is fixed to an inner surface of the first curler 51. The first pressing cuff 52, by being inflated, presses the back side of the wrist 300 and presses the back plate 53 and the sensing cuff 54 toward the wrist 300.

The first pressing cuff 52 includes, for example, one or more of the air bags 71, the flow path body 72 provided at an end portion of the air bag 71, and the connection portion 73 such as a nipple provided in the flow path body 72 and connected to the fluid circuit 24. Here, the air bag 71 is a bag-like structure and, in the present embodiment, the blood pressure measurement device 1 is configured to use air by the pump 14, and thus the present embodiment will be described using the air bag. However, in a case in which a fluid other than air is used, the bag-like structure need only be any fluid bag inflated by the fluid. The air bag 71 is formed in a rectangular bag shape that is long in one direction.

The air bag 71 is made by forming a plurality of sheet members into a bag shape by thermal welding or the like. For example, when a configuration is adopted in which the first pressing cuff 52 is configured to include a plurality of the air bags 71, the plurality of air bags 71 are layered, integrally formed by welding or the like, and fluidly continuous. The sheet members forming the air bag 71 are made of a thermoplastic elastomer, for example. The thermoplastic elastomer is, for example, TPU.

The flow path body 72 is, for example, integrally provided at a part of one edge portion of the air bag 71 in a longitudinal direction. The flow path body 72 is formed by a portion of two sheet members of the plurality of sheet members forming the air bag 71. Further, the flow path body 72 is formed in a shape that is long in one direction with a width smaller than a width of the air bag 71 in a lateral direction. The flow path body 72 is integrally provided with the connection portion 73 at a tip end. The flow path body 72 is connected to the fluid circuit 24 via the connection portion 73 and constitutes a flow path between the fluid circuit 24 and the air bag 71. A thickness of the flow path body 72 at the time of inflation is smaller than a thickness of the air bag 71 at the time of inflation.

For example, one connection portion 73 is provided. The one connection portion 73 is connected to the fluid circuit 24 via one hole portion 31b3 formed in the bottom portion 31b.

Note that, for example, the flow path body 72 extends from one end side to the other end side of the bottom portion 31b in the opposing direction of the pair of loop portions 31c adjacent to the projection portion 31b1 so as to avoid the projection portion 31b1 formed at the bottom portion 31b, and the connection portion 73 is connected to one hole portion 31b3 among the plurality of hole portions 31b3 formed at an end portion of the bottom portion 31b on the side where the second cuff structure 5 projects.

The back plate 53 is fixed to the surface of the first pressing cuff 52 on the wrist 300 side by double-sided tape, an adhesive, or the like. The back plate 53 is formed of a resin material. The back plate 53 is formed in a rectangular plate shape that is long in one direction, for example. Note that the back plate 53 may, for example, be configured to be divided, that is, may be formed by aligning a plurality of small rectangular pieces in one direction. The back plate 53 has shape followability.

This “shape followability” refers to a function in which the back plate 53 can be deformed in such a manner as to follow the shape of a contacted portion of the wrist 300 to be disposed. This contacted portion of the wrist 300 refers to a region of the wrist 300 that comes into contact with the back plate 53. This contact includes both direct contact with the back plate 53 and indirect contact with the back plate 53 with the sensing cuff 54 interposed therebetween.

The sensing cuff 54 is fixed to a main surface of the back plate 53 on the wrist side. The sensing cuff 54 comes into contact with a region of the wrist 300 where the arteries 311, 312 are present, directly or indirectly with a cover or the like interposed therebetween. The sensing cuff 54 is formed into a rectangular shape that is long in one direction. Note that a configuration may be adopted in which the sensing cuff 54 comes into contact with a region of the wrist 300 where one of the arteries 311, 312 is present. The sensing cuff 54 is the same size as the first pressing cuff 52 or smaller than the first pressing cuff 52 in the longitudinal direction. Further, the sensing cuff 54 is the same size as the first pressing cuff 52 or smaller than the first pressing cuff 52 in the lateral direction. The sensing cuff 54 has the same shape as that of the back plate 53 or is smaller than the back plate 53 or is larger than the back plate 53 in the longitudinal direction and a width direction of the back plate 53. The sensing cuff 54 compresses the region of the wrist on the palm side where the artery is present by being inflated. The sensing cuff 54 is pressed by the inflated first pressing cuff 52 toward the living body with the back plate 53 interposed therebetween.

As a specific example, the sensing cuff 54 includes the air bag 81, the flow path body 82 fluidly connected to the air bag 81, and the connection portion 83 such as a nipple provided at the flow path body 82. The air bag 81 and the flow path body 82 are made by forming a plurality of sheet members into a bag shape by thermal welding or the like. The sheet members forming the air bag 81 and the flow path body 82 are formed of, for example, a thermoplastic elastomer. The thermoplastic elastomer is, for example, TPU.

Here, the air bag 81 is a bag-like structure and, in the present embodiment, a configuration is adopted in which the blood pressure measurement device 1 uses air with the pump 14, and thus the present embodiment will be described using the air bag. However, in a case in which a fluid other than air is used, the bag-like structure may be a fluid bag or the like that is inflated by the fluid. The air bag 81 is constituted in a rectangular shape that is long in one direction.

The flow path body 82 is, for example, integrally provided at a part of one edge portion of the air bag 81 in the longitudinal direction. The flow path body 82 is formed by a portion of two sheet members of the plurality of sheet members forming the air bag 81. Further, the flow path body 82 is formed in a shape that is long in one direction with a width smaller than a width of the air bag 81 in the lateral direction. The flow path body 82 is integrally provided with the connection portion 83 at a tip end. The flow path body 82 is connected to the fluid circuit 24 via the connection portion 83 and constitutes a flow path between the fluid circuit 24 and the air bag 81. A thickness of the flow path body 82 at the time of inflation is smaller than a thickness of the air bag 81 at the time of inflation.

A plurality of the connection portions 83 are provided. The plurality of connection portions 83 are connected to the plurality of hole portions 31b3 formed at an end portion of the bottom portion 31b on the side from which the first cuff structure 4 projects. The plurality of connection portions 83 are connected to the fluid circuit 24.

Hereinafter, a specific example of the second cuff structure 5 will be described with reference to FIG. 1 to FIG. 3 and FIG. 5. As illustrated in FIG. 1 to FIG. 3 and FIG. 5, the second cuff structure 5 includes a second curler 61 and the second pressing cuff 62. The second cuff structure 5 is configured by sequentially layering the second curler 61 and the second pressing cuff 62 toward the wrist 300.

The second curler 61 is, for example, fixed, at one end side, to the bottom portion 31b provided on the wrist 300 side of the case 11. The second curler 61 is formed in a band-like shape that curves in a shape following the circumferential direction of the wrist 300. The second curler 61 is constituted by a resin material. The second curler 61 is formed of a material having a low hardness appropriate to provide flexibility and shape retainability. Here, “flexibility” refers to deformation of the shape of the second curler 61 in the radial direction at the time of application of an external force from the band 6 to the second curler 61. “Shape retainability” refers to the ability of the second curler 61 to maintain a pre-imparted shape when no external force is applied to the second curler 61. That is, the second curler 61 is formed of a resin material having a hardness that does not undergo compression deformation or is substantially without compression deformation but allows elastic deformation such as bending deformation in which the shape, particularly the curvature of a curved portion, changes. Accordingly, the second curler 61 is formed to be elastically deformable so as to be bent and deformed by application of an external force and to increase or decrease the internal space in which the wrist 300 is disposed in accordance with the shape of the wrist to which second curler 61 is attached. For example, the second curler 61 is formed of a thermoplastic polyurethane resin (hereinafter referred to as TPU) or a polypropylene resin.

Further, the second curler 61 is, on one end side, fixed to the case 11. Further, the second curler 61 is formed to such a length so as to face at least one of the two arteries 311, 312 and overlap the first cuff structure 4 at an end portion side when the blood pressure measurement device 1 is attached to the wrist 300 having the longest circumference among the users who are expected to attach the device. Here, one of the two arteries 311, 312 is the radial artery 311 and the other is the ulnar artery 312. Preferably, the second curler 61 is formed to such a length so as to face the two arteries 311, 312 when the blood pressure measurement device 1 is attached to the wrist 300 having the longest circumference among the users who are expected to attach the device. Further, the second curler 61 is set to such a length that the other end of the second curler 61 does not come into contact with the device body 3 when the blood pressure measurement device 1 is attached to the wrist 300 having the shortest circumference among the users who are expected to attach the device. In the second curler 61, for example, a portion extending from the case 11 of the device body 3 is curved with a predetermined radius of curvature so as to follow the shape of the side of one wrist 300 of the left and right wrists 300 or the palm side of the wrist 300.

The second pressing cuff 62 is fixed to an inner peripheral surface of the second curler 61 by double-sided tape, an adhesive, thermal welding, or the like. The second pressing cuff 62 is fluidly connected to the pump 14 through the fluid circuit 24. The second pressing cuff 62, at one main surface, is fixed to an inner surface of the second curler 61. The second pressing cuff 62, by being inflated, presses the opposing wrist 300 and presses the overlapped first cuff structure 4 toward the wrist 300. A thickness of the second pressing cuff 62 when inflated is the same as a thickness of the first pressing cuff 52 or greater than the thickness of the first pressing cuff 52 when inflated.

The second pressing cuff 62 includes, for example, one or more of the air bags 91, the flow path body 92 provided at an end portion of the air bag 91, and the connection portion 93 such as a nipple provided in the flow path body 92 and connected to the fluid circuit 24. Here, the air bag 91 is a bag-like structure, and in the present embodiment, a configuration is adopted in which the blood pressure measurement device 1 uses air with the pump 14, and thus the present embodiment will be described using the air bag. However, in a case in which a fluid other than air is used, the bag-like structure need only be a fluid bag that is inflated by a fluid. The air bag 91 is formed in a rectangular bag shape that is long in one direction.

The air bag 91 is made by forming a plurality of sheet members into a bag shape by thermal welding or the like. For example, when a configuration is adopted in which the second pressing cuff 62 includes a plurality of the air bags 91, the plurality of air bags 91 are layered, integrally formed by welding or the like, and fluidly continuous. The sheet members forming the air bag 91 are made of a thermoplastic elastomer, for example. The thermoplastic elastomer is, for example, TPU.

The flow path body 92 is, for example, integrally provided at a part of one edge portion of the air bag 91 in the longitudinal direction. The flow path body 92 is formed by a portion of two sheet members of the plurality of sheet members forming the air bag 91. Further, the flow path body 92 is formed into a shape that is long in one direction with a width smaller than a width of the air bag 91 in the lateral direction. The flow path body 92 is integrally provided with the connection portion 93 at a tip end. The flow path body 92 is connected to the fluid circuit 24 via the connection portion 93 and constitutes a flow path between the fluid circuit 24 and the air bag 91. A thickness of the flow path body 92 at the time of inflation is smaller than a thickness of the air bag 91 at the time of inflation.

For example, a plurality of the connection portions 93 are provided. The plurality of connection portions 93 are connected to the fluid circuit 24 via the plurality of hole portions 31b3 formed in the bottom portion 31b.

Note that, for example, the connection portions 93 are connected to any one or more hole portions 31b3 among the plurality of hole portions 31b3 formed in the end portion of the bottom portion 31b on the side from which the second cuff structure 5 projects.

The first pressing cuff 52 and the second pressing cuff 62 of the first cuff structure 4 and the second cuff structure 5 thus configured are formed to such a length that, when the blood pressure measurement device 1 is attached to the wrist 300 having the maximum circumferential length, the inflated portion of the air bag 71 of the first pressing cuff 52 can cover at least one of the two arteries 311, 312, and the air bag 91 of the second pressing cuff 62 can overlap the air bag 71. Further, the air bag 91 of the second pressing cuff 62 is formed to such a length that, when the blood pressure measurement device 1 is attached to the wrist 300 having the maximum circumferential length and the air bag 91 overlaps the air bag 71 of the first pressing cuff 52, an end edge of the air bag 91 on the side opposite to the flow path body 92 does not overlap the air bag 71 of the first pressing cuff 52 on the artery covered by the air bag 71 of the first pressing cuff 52. That is, the air bag 91 of the second pressing cuff 62 is set to such a length that, when the blood pressure measurement device 1 is attached to the wrist 300 having the maximum circumferential length and the air bag 91 overlaps the air bag 71 of the first pressing cuff 52, the inflated portion of the air bag 91 of the second pressing cuff 62 overlaps the air bag 71 of the first pressing cuff 52 on the artery covered by the air bag 71 of the first pressing cuff 52.

The band 6 is formed in a belt-like shape on an outer peripheral side, on the side opposite to the wrist 300, of the first cuff structure 4 and the second cuff structure 5. The band 6 is not fixed to the first cuff structure 4 and the second cuff structure 5. The band 6 is, at one end portion, fixed to one loop portion 31c. Further, the band 6 includes a pair of hook-and-loop fasteners 6a in which a hook is formed on one side and a loop is formed on the other side, and the band 6 with an end portion side inserted into the other loop portion 31c is fixed by engaging the hook and the loop with each other. Further, the band 6 includes, at the other end portion, a grip 6b that facilitates gripping by the user.

For example, the band 6 is fixed to the loop portion 31c formed on the side from which the second cuff structure 5 extends, is inserted into the loop portion 31c formed on the side from which the first cuff structure 4 extends, and is then folded back. The band 6 has such a length that, when the blood pressure measurement device 1 is attached to the wrist 300 having the maximum circumferential length at which attachment is expected, the band 6 can be inserted into the loop portion 31c provided at the outer case 31. Further, the hook-and-loop fastener 6a is provided at the band 6 at such a length and in such an arrangement that the band 6 can be folded back and fastened at the wrists 300 having the maximum circumferential length and the minimum circumferential length at which attachment is expected. The end portion of the band 6 folded back at the loop portion 31c is pulled in a direction away from the loop portion 31c, attaching the blood pressure measurement device 1 onto the wrist 300 in a state in which the first cuff structure 4 and the second cuff structure 5 are wound around the wrist 300 as illustrated in FIG. 3. The band 6 is fixed by the hook-and-loop fastener 6a, restricting the first pressing cuff 52 and the second pressing cuff 62, when inflated, from expanding outwardly away toward a side opposite to the wrist, and thus making it possible for the first pressing cuff 52 and the second pressing cuff 62 to compress the wrist. This allows the blood pressure measurement device 1 to perform blood pressure measurement by a known oscillometric method.

Next, an example of a fluid configuration of the fluid circuit 24 of the blood pressure measurement device 1 thus configured will be described with reference to FIG. 10 to FIG. 12. Note that, in this example, one connection portion 73 of the first pressing cuff 52 is provided, three connection portions 83 of the sensing cuff 54 are provided, and three connection portions 93 of the second pressing cuff 62 are provided.

First, a configuration of the flow path plate unit (fluid circuit) 24 will be described with reference to FIG. 10 to FIG. 12. The flow path plate unit 24 includes the flow path plate 24a and the two flow path resistors 24b1, 24b2 provided in the flow path plate 24a.

A plurality of internal flow paths are formed in the flow path plate 24a. As a specific example, the flow path plate 24a includes a first internal flow path 241, the second internal flow path 242, a third internal flow path 243, a fourth internal flow path 244, a fifth internal flow path 245, a sixth internal flow path 246, and a seventh internal flow path 247.

The first internal flow path 241 connects the pump 14 and the first pressing cuff 52 as well as the second pressing cuff 62. The first internal flow path 241 is a flow path branched in three directions. The second internal flow path 242 connects the second pressing cuff 61 and the valve 16. The third internal flow path 243 connects the second pressing cuff 61 and the first flow path resistor 24b1. The fourth internal flow path 244 connects the first flow path resistor 24b1 and the sensing cuff 54. The fifth internal flow path 245 connects the sensing cuff 54 and the pressure sensor 17. The sixth internal flow path 246 connects the sensing cuff 54 and the second flow path resistor 24b2. The seventh internal flow path 247 connects the second flow path resistor 24b2 and the atmosphere, and opens a secondary side of the second flow path resistor 24b2 to the atmosphere.

The plurality of internal flow paths 241 to 247 are formed in the flow path plate 24a as independent flow paths.

The flow path plate 24a includes, for example, a first port 24a1 connecting the pump 14, a second port 24a2 connecting the valve 16, a third port 24a3 connecting the pressure sensor 17, and a fourth port 24a4 open to the atmosphere. For example, the first port 24a1 to the fourth port 24a4 are provided in an upper surface of the flow path plate 24a, that is, in a main surface of the flow path plate 24a on the windshield 32 side.

The first port 24a1 is formed in one end portion of three end portions of the first internal flow path 241. The second port 24a2 is formed at one end portion of the second internal flow path 242. The third port 24a3 is formed at one end portion of the fifth internal flow path 245. The fourth port 24a4 is formed at one end portion of the seventh internal flow path 247.

The flow path plate 24a includes, for example, the plurality of ports 24c selectively connected to the cuffs of the first cuff structure 4 and the second cuff structure 5 and the testers or sealed. The plurality of ports 24c are disposed facing the plurality of hole portions 31b3 formed in the bottom portion 31b of the outer case 31.

For example, the plurality of ports 24c include one fifth port 24cl connectable to the connection portion 73 of the first pressing cuff 52, three sixth ports 24c2 connectable to the three connection portions 93 of the second pressing cuff 62, and three seventh ports 24c3 connectable to the three connection portions 83 of the sensing cuff 54. For example, the fifth port 24cl to the seventh ports 24c3 are provided in a lower surface of the flow path plate 24a, that is, in a main surface of the flow path plate 24a on the bottom portion 31b side.

The fifth port 24cl is formed in, among the three end portions of the first internal flow path 241, an end portion different from the end portion to which the first port 24a1 is connected. The three sixth ports 24c2 are respectively formed in, among the three end portions of the first internal flow path 241, an end portion different from the end portions to which the first port 24a1 and the fifth port 24cl are connected, the other end portion of the second internal flow path 242, and the other end portion of the third internal flow path 243. The three seventh ports 24c3 are formed in the other end portion of the fourth internal flow path 244, the other end portion of the fifth internal flow path 245, and the other end portion of the sixth internal flow path 246.

The plurality of ports 24c are formed so as to be selectively connectable to the connection portions 73, 83, 93 of the respective cuffs 52, 54, 62 and various testers. Further, the plurality of ports 24c are sealably formed.

Next, as the blood pressure measurement device 1, a configuration of the fluid circuit 24 to which the first cuff structure 4 and the second cuff structure 5 are connected will be described with reference to FIG. 11. The first pressing cuff 52 is fixed to the fifth port 24cl at the connection portion 73, and the second pressing cuff 62 is fixed to one sixth port 24c2 at the connection portion 93. Thus, the first pressing cuff 52 and the second pressing cuff 62 are connected to the pump 14 through the first internal flow path 241 of the flow path plate 24a.

The second pressing cuff 62 is connected to the valve 16 through the second internal flow path 242 of the flow path plate 24a by the connection portion 93 being fixed to one sixth port 24c2. Further, the second pressing cuff 62 is connected to the first flow path resistor 24b1 through the third internal flow path 243 of the flow path plate 24a by the connection portion 93 being fixed to one sixth port 24c2.

The sensing cuff 54 is connected to the first flow path resistor 24b1 through the fourth internal flow path 244 of the flow path plate 24a by the connection portion 83 being fixed to one seventh port 24c3. Thus, the sensing cuff 54 is connected to the second pressing cuff 62 through the first flow path resistor 24b1. Further, the sensing cuff 54 is connected to the pressure sensor 17 through the fifth internal flow path 245 of the flow path plate 24a by the connection portion 83 being fixed to one seventh port 24c3. Further, the sensing cuff 54 is connected to the second flow path resistor 24b2 through the sixth internal flow path 246 of the flow path plate 24a by the connection portion 83 being fixed to one seventh port 24c3, thereby connecting the sensing cuff 54 to the atmosphere through the second flow path resistor 24b2. Here, the first flow path resistor 24b1 between the first pressing cuff 52 and the sensing cuff 54 and the second flow path resistor 24b2 between the sensing cuff 54 and the atmosphere are set to flow path resistances at which the first pressing cuff 52 and the sensing cuff 54 have a desired difference in pressure.

Next, a configuration of the fluid circuit 24 to which the blood pressure measurement device 1 and the testers are connected when testing each component will be described with reference to FIG. 12.

One of the fifth port 24cl or one sixth port 24c2 connected to the first internal flow path 241 is sealed, and the other is connected to a pump tester 401 that tests a performance of the pump 14. Note that the example illustrated in FIG. 12 illustrates an example in which the fifth port 24cl is sealed and the pump tester 401 is connected to the sixth port 24c2. Thus, with the pump tester 401 connected only to the pump 14 through the first internal flow path 241, it is possible to test the pump 14.

The sixth port 24c2 connected to the second internal flow path 242 is connected to a valve tester 402 that tests the valve 16. Thus, with the valve tester 402 connected only to the valve 16 through the second internal flow path 242, it is possible to test the valve 16.

The sixth port 24c2 connected to the third internal flow path 243 is connected to a first flow path resistor tester 403 that tests the first flow path resistor 24b1, and the seventh port 24c3 connected to the fourth internal flow path 244 is opened to the atmosphere. Accordingly, the first flow path resistor tester 403 is connected to the atmosphere through the first flow path resistor 24b1 by the third internal flow path 243 and the fourth internal flow path 244, making it possible to test the first flow path resistor 24b1.

The seventh port 24c3 connected to the fifth internal flow path 245 is connected to a sensor tester 404 that tests the pressure sensor 17. Thus, with the sensor tester 404 connected only to the pressure sensor 17 through the fifth internal flow path 245, it is possible to test the pressure sensor 17.

The seventh port 24c3 connected to the sixth internal flow path 246 is connected to a second flow path resistor tester 405 that tests the second flow path resistor 24b2, and the fourth port 24a4 connected to the seventh internal flow path 247 is opened to the atmosphere. Thus, with the second flow path resistor tester 405 connected to the atmosphere through the second flow path resistor 24b2 by the sixth internal flow path 246 and the seventh internal flow path 247, it is possible to test the second flow path resistor 24b2.

Thus, the various testers 401 to 405 are respectively connected to testing targets through the internal flow paths 241 to 247, making it possible to test the testing targets independently. Further, the various testers 401 to 405 need only be connected to the plurality of ports 24c to which the cuffs 52, 54, 62 are connected, simplifying connection. That is, the plurality of ports 24c are selectively connectable to the respective cuffs 52, 54, 62 and the respective testers 401 to 405, facilitating the attachment/detachment and the sealing of the respective cuffs 52, 54, 62 and the respective testers 401 to 405 during blood pressure measurement and testing.

Next, an example of the power transmission device 100 that transmits power to the charging circuit unit 21 of the device body 3 will be described.

As illustrated in FIG. 4, the power transmission device 100 includes a power source 101, a power transmission unit 102, the antenna unit 103, and the power transmission terminal 104. Note that a configuration may be adopted in which the power transmission device 100 includes one of the antenna unit 103 or the power transmission terminal 104. The power source 101 is, for example, an AC adapter connected to a commercial power source or the like. The power source 101 converts AC power input from a commercial power source into DC power and supplies the DC power to the power transmission unit 102.

The power transmission unit 102 generates AC power as transmission power from the DC power supplied from the power source 101, and supplies the AC power to the antenna unit 103. For example, the power transmission unit 102 generates AC power having a frequency that is the same or substantially the same as a resonance frequency of the power transmission resonance circuit of the antenna unit 103.

The antenna unit 103 is, for example, a transmitter coil of a power transmission resonance circuit. A power transmission surface of the antenna unit 103 is formed into a planar shape. The antenna unit 103 transmits power to the antenna unit 211 of the device body 3. The antenna unit 103 includes, for example, a resonance capacitor and constitutes a power transmission resonance circuit.

The power transmission terminal 104 comes into contact with the charging terminal 214 provided in the device body 3 and is formed so as to be fixable to the charging terminal 214.

In the blood pressure measurement device 1 thus configured, the plurality of internal flow paths 241 to 247 formed in the flow path plate 24a of the flow path plate unit (fluid circuit) 24 respectively connect the pump 14 and the first pressing cuff 52 as well as the second pressing cuff 62, the valve 16 and the second pressing cuff 62, the second pressing cuff 62 and the first flow path resistor 24b1, the first flow path resistor 24b1 and the sensing cuff 54, the pressure sensor 17 and the sensing cuff 54, the sensing cuff 54 and the second flow path resistor 24b2, and the second flow path resistor 24b2 and the atmosphere. Further, the internal flow paths 241 to 247 are independent from one another. Thus, when the blood pressure measurement device 1 is in a usable state in which the blood pressure can be measured, the flow path plate unit 24 can connect the pump 14, the valve 16, and the pressure sensor 17 serving as components to the cuffs 52, 54, 62. Further, at the time of manufacture or at the time of maintenance of the blood pressure measurement device 1, the components 14, 16, 17 and the testers 401 to 405 can be connected to each other in a testing state in which the components 14, 16, 17 are tested, and can be sealed or opened to the atmosphere when testing is not performed. Thus, the blood pressure measurement device 1 can individually test the pump 14, the valve 16, and the pressure sensor 17 provided in the flow path plate unit 24 without increasing a size of the flow path plate 24a in which the flow path resistors 24b1, 24b2 are provided, that is, the flow path plate unit 24.

Further, in the blood pressure measurement device 1, the second pressing cuff 62 and the first flow path resistor 24b1, the first flow path resistor 24b1 and the sensing cuff 54, the sensing cuff 54 and the second flow path resistor 24b2, and the second flow path resistor 24b2 and the atmosphere can be connected by the third internal flow path 243, the fourth internal flow path 244, the sixth internal flow path 246, and the seventh internal flow path 247, respectively. Thus, the blood pressure measurement device 1 can also test the first flow path resistor 24b1 and the second flow path resistor 24b2 by connecting the first flow path resistor tester 403 and the second flow path resistor tester 405 to the third internal flow path 243 and the sixth internal flow path 246, respectively, and opening the fourth internal flow path 244 to the atmosphere.

Further, a configuration is adopted in which the flow path plate 24a includes the plurality of ports 24c selectively connectable to the connection portions 73, 83, 93 of the cuffs 52, 54, and 62 and the testers 401 to 405. That is, in the flow path plate 24a, the plurality of ports 24c can be used for blood pressure measurement as well as testing without providing ports to which the cuffs 52, 54, 62 and the testers 401 to 405 are respectively connected, making it possible to prevent an enlargement in shape and facilitating sealing and atmosphere release. That is, the cuffs 52, 54, 62, the testers 401 to 405, and sealing plugs are selectively connectable to the ports 24c from the hole portions 31b3 in a state in which the cuff cover 33 is removed, making it possible to simplify the internal flow paths 241 to 247 and thus impart high workability to the blood pressure measurement device 1.

Further, in the blood pressure measurement device 1, the first cuff structure 4 and the second cuff structure 5 extending from the device body 3 and fluidly connected to each other are respectively disposed on both sides of the device body 3 in one direction. Further, the device body 3 includes, at the bottom portion 31b of the case 11 that is the rear surface side, the projection portion (sensor unit) 31b1 as a space in which at least one of the biometric sensors 20 or the charging terminal 214 is disposed at a position separated from the first cuff structure 4 and the second cuff structure 5, for example, on a center side of the bottom portion 31b. Accordingly, the first cuff structure 4, the second cuff structure 5, and at least one of the biometric sensors 20 or the charging terminal 214 can each be disposed on the rear surface side of the device body 3.

Further, the first cuff structure 4 and the second cuff structure 5 are fluidly connected to each other through the fluid circuit 24 including the flow path plate 24a. Note that the first cuff structure 4 and the second cuff structure 5 may directly fluidly connect the first pressing cuff 52 and the second pressing cuff 62. With these configurations, even when the first cuff structure 4 and the second cuff structure 5 are provided and fixed to the case 11 away from each other in one direction, it is possible to fluidly connect the first cuff structure 4 and the second cuff structure 5.

Further, when the blood pressure measurement device 1 is attached to the wrist 300 having the maximum circumferential length expected for attachment, the air bag 71 of the first pressing cuff 52 covers the region where at least one artery of the two arteries 311, 312 is present, and the air bag 91 of the second pressing cuff 62 can overlap at least part of the air bag 71 of the first pressing cuff 52.

Thus, the wrist 300 can be pressed by the first pressing cuff 52 and the second pressing cuff 62 and, even if the air bag 71 of the first pressing cuff 52 and the air bag 91 of the second pressing cuff 62 are not disposed at the rear surface of the device body 3, a sufficient pressing force for compressing the artery can be secured.

Thus, when the blood pressure measurement device 1 is attached to the wrist 300 having the maximum circumferential length expected for attachment, the air bag 71 of the first pressing cuff 52 and the air bag 91 of the second pressing cuff 62 can overlap by a predetermined length. Further, when the blood pressure measurement device 1 is attached to the wrist 300 having the minimum circumferential length, the first cuff structure 4 and the second cuff structure 5 each have such a length so as to not come into contact with the device body 3. Further, the first cuff structure 4 and the second cuff structure 5 are not directly joined to the band 6.

With these configurations, the blood pressure measurement device 1, with the air bags 71, 91 of the first pressing cuff 52 and the second pressing cuff 62 being inflated in an overlapping manner, makes it possible to suitably press the artery by the first pressing cuff 52 and the second pressing cuff 62. Thus, the blood pressure measurement device 1 makes it possible to dispose the biometric sensors 20 on the rear surface side of the device body 3 and, even if the air bags 71, 91 of the first pressing cuff 52 and the second pressing cuff 62 are not disposed at the rear surface of the device body 3, measure blood pressure with favorable accuracy.

Further, the case 11 includes the vent 31b6 covered with the waterproof moisture-permeable sheet 31b7 at the bottom portion 31b, facilitating ventilation in the case 11. Further, the vent 31b6 and the first cuff structure 4 as well as the second cuff structure 5 are disposed at positions separated from each other in a portion of the bottom portion 31b other than the projection portion 31b1, making it possible to prevent the vent 31b6 from being blocked by the first cuff structure 4 and the second cuff structure 5. Further, the vent 31b6 is provided at a position covered with the cuff cover 33 disposed spaced apart from the bottom portion 31b, thereby protecting the vent 31b6 with the cuff cover 33. Thus, the blood pressure measurement device 1 can prevent dirt from adhering to the vent 31b6 and the waterproof moisture-permeable sheet 31b7, making it possible to prevent impairment of the ventilation function.

Further, the first cuff structure 4 and the second cuff structure 5 are covered with the cuff cover 33 at end portions disposed at the bottom portion 31b of the case 11, and are thus restricted in movement in a direction away from the bottom portion 31b by the cuff cover 33. This makes it possible to prevent the first cuff structure 4 and the second cuff structure 5 from being readily detached from the device body 3.

Further, a configuration is adopted in which the blood pressure measurement device 1 is provided with the various sensors 20a to 20c as the biometric sensors 20 and the charging terminal 214 at the bottom portion 31b of the device body 3. Thus, the device body 3 enables simplification of wiring and the like, increasing a degree of freedom in arrangement, by not requiring the biometric sensors 20 and the charging terminal 214 to be provided from the substrate 25 provided inside the case 11 to a position far from the device body. Further, the biometric sensors 20 are provided in the case 11 having high rigidity, and can stably come into contact with the wrist 300 when the blood pressure measurement device 1 is attached to the wrist 300, making it possible to suitably acquire biological information by the biometric sensors 20. Further, the charging terminal 214 is provided at the bottom portion 31b and thus, when the blood pressure measurement device 1 is removed from the wrist 300, the charging terminal 214 is exposed to the outside, facilitating charging using the charging terminal 214.

Further, in the blood pressure measurement device 1, the first pressing cuff 52 and the second pressing cuff 62 are fluidly connected through the fluid circuit 24 provided inside the device body 3, making it possible simplify the arrangement of the first pressing cuff 52 and the second pressing cuff 62 at the bottom portion 31b.

The blood pressure measurement device 1 thus configured makes it possible to individually test, by the testers 401 to 405, the pump 14, the valve 16, the pressure sensor 17, the first flow path resistor 24b1, and the second flow path resistor 24b2, which are components provided in the flow path plate unit 24, without increasing the size of the flow path plate unit 24.

Note that the present invention is not limited to the embodiment described above. For example, in the above-described example, an example is described in which the flow path plate unit 24 that is the fluid circuit includes the first internal flow path 241 connecting the pump 14, the first pressing cuff 52, and the second pressing cuff 62, and the third internal flow path 243 connecting the second pressing cuff 62 and the first flow path resistor 24b1 as independent flow paths, and includes the fourth internal flow path 244 connecting the first flow path resistor 24b1 and the sensing cuff 54, and the sixth internal flow path 246 connecting the sensing cuff 54 and the second flow path resistor 24b2 as independent flow paths.

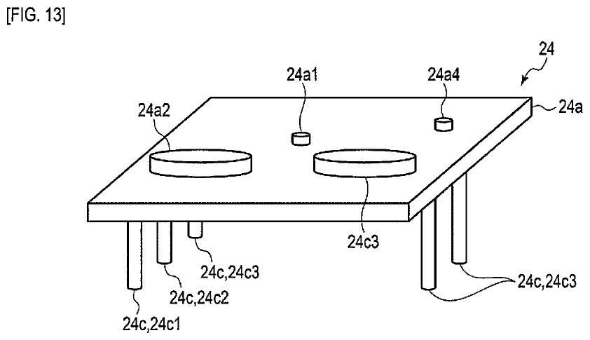

However, as illustrated in FIG. 13 to FIG. 16, a configuration may be adopted in which the flow path plate unit 24 is provided with an eighth internal flow path 241, 243 obtained by integrating the first internal flow path 241 and the third internal flow path 243, and a ninth internal flow path 244, 246 obtained by integrating the fourth internal flow path 244 and the sixth internal flow path 246.

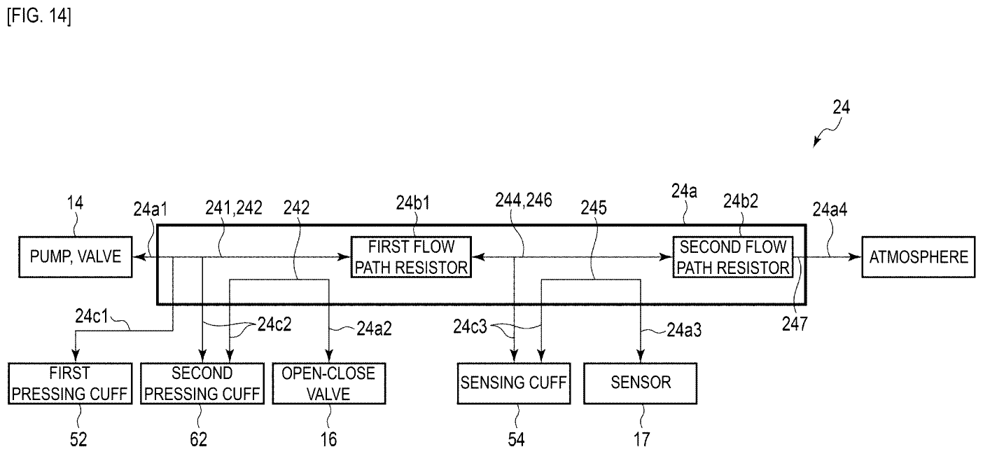

The blood pressure measurement device 1 having such a configuration is provided with one connection portion 73 of the first pressing cuff 52, two connection portions 83 of the sensing cuff 54, and two connection portions 93 of the second pressing cuff 62. That is, as illustrated in FIG. 14, two connection portions 83 of the sensing cuff 54 and two connection portions 93 of the second pressing cuff 62 can be provided and, as illustrated in FIG. 13 and FIG. 14, two sixth ports 24c2 and two seventh ports 24c3 can be provided.

For example, as illustrated in FIG. 14, the eighth internal flow path 241, 243 is connected to the pump 14, the first pressing cuff 52, the second pressing cuff 62, and the first flow path resistor 24b1 through the first port 24a1, the fifth port 24cl, and the sixth port 24c2. Further, as illustrated in FIG. 14, the ninth internal flow path 244, 246 is connected to the first flow path resistor 24b1, the second flow path resistor 24b2, and the sensing cuff 54 through the seventh port 24c3.

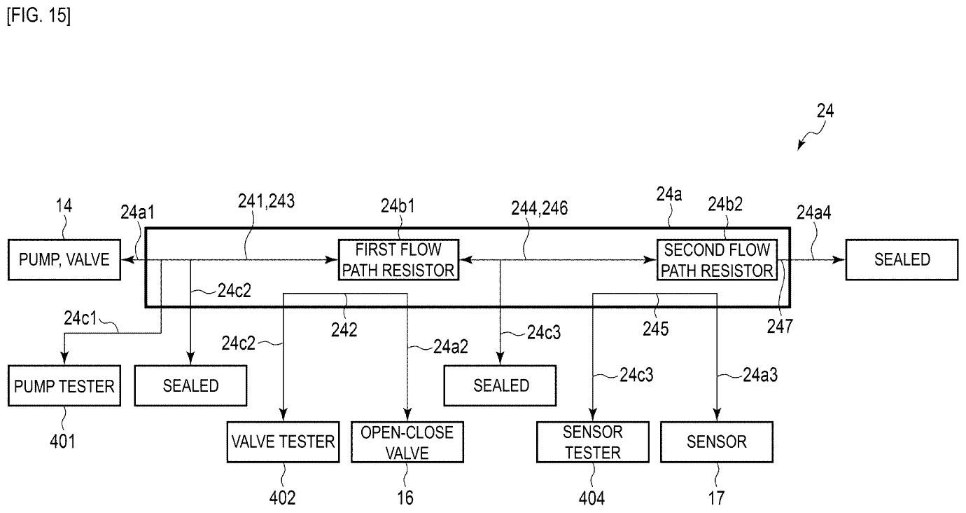

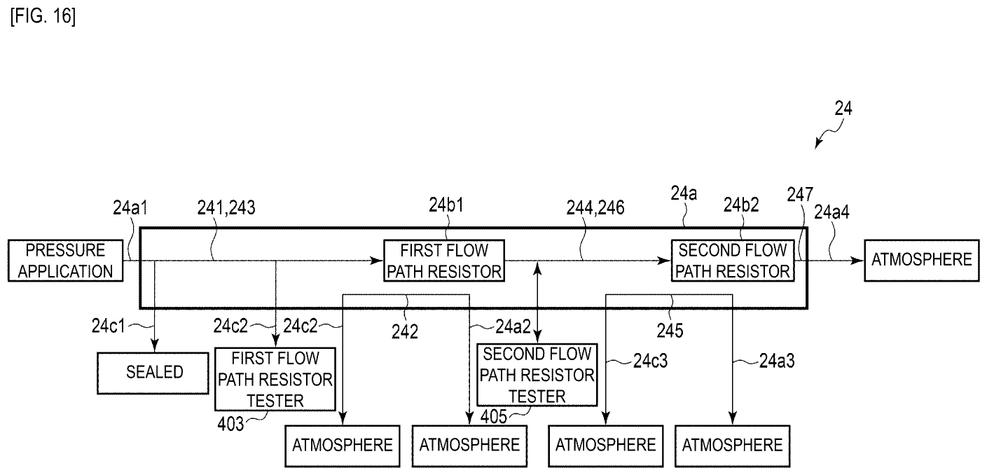

Further, during the testing of the pump 14, as illustrated in FIG. 15, the pump tester 401 need only be connected to the fifth port 24cl and the one sixth port 24c2 connected to the eighth internal flow path 241, 243, and the other of the sixth ports 24c2, the seventh port 24c3 connected to the ninth internal flow path 244, 246, and the fourth port 24a4 opened to the atmosphere need only be sealed. Further, during testing of the first flow path resistor 24b1 and the second flow path resistor 24b2, as illustrated in FIG. 16, the first flow path resistor tester 403 need only be connected to the fifth port 24cl and the one sixth port 24c2 connected to the eighth internal flow path 241, 243, the second flow path resistor tester 405 need only be connected to the other of the sixth ports 24c2 and the seventh port 24c3 connected to the ninth internal flow path 244, 246, the pump 14 need only apply pressure from the first port 24a1, and the fourth port 24a4 need only be opened to the atmosphere.

According to the blood pressure measurement device 1 having such a configuration, it is possible to reduce the number of connection points between the flow path plate unit 24 and the cuffs 54, 62, and to test the pump 14, the valve 16, and the pressure sensor 17. Further, the resistance values due to the first flow path resistor 24b1 and the second flow path resistor 24b2 can be obtained from a pressure difference measured by the first flow path resistor tester 403 and the second flow path resistor tester 405.

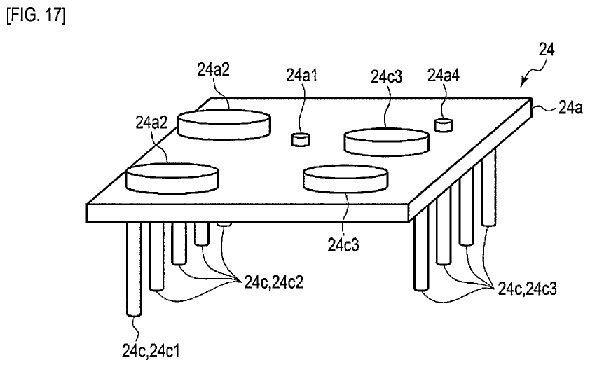

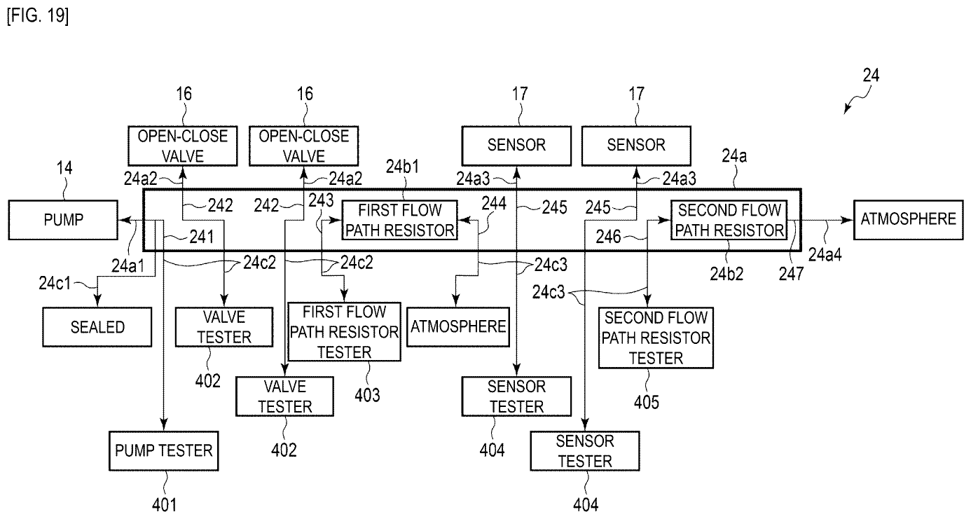

Further, in the example described above, an example is described in which the pump 14 has a configuration including the valve mechanism as an open-close valve. However, the configuration is not limited thereto, and the pump 14 may have a configuration not including the valve mechanism. For example, FIG. 17 to FIG. 19 illustrate an example of the flow path plate unit 24 in the case of adopting a configuration in which the pump 14 does not include a valve mechanism.

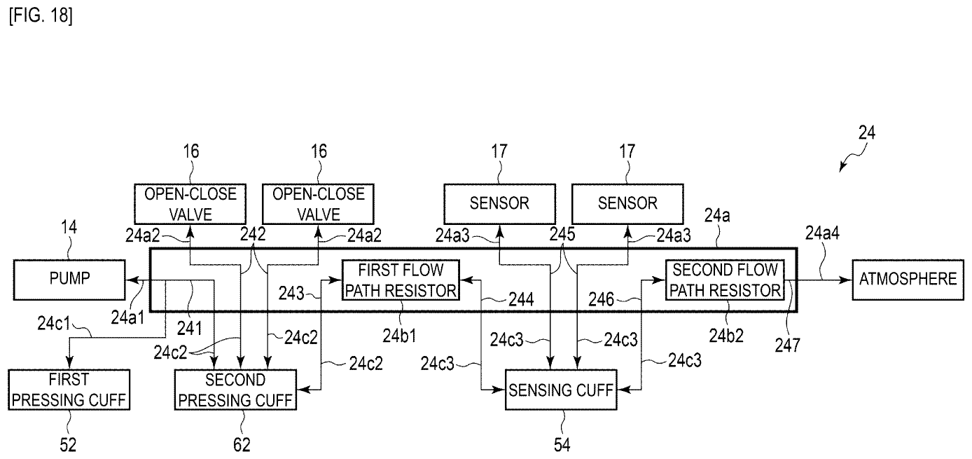

For example, in the case of adopting a configuration in which the pump 14 does not include a valve mechanism, the blood pressure measurement device 1 includes, for example, two valves 16 and two pressure sensors 17. Further, as illustrated in FIG. 17 and FIG. 18, the flow path plate unit 24 includes two second ports 24a2 and two third ports 24a3, includes two independent second internal flow paths 242 and two independent fifth internal flow paths 245, and includes four sixth ports 24c2 and four seventh ports 24c3. Further, the sensing cuff 54 includes four connection portions 83, and the second pressing cuff 62 includes four connection portions 93.

Further, during testing, in the blood pressure measurement device 1, the valve tester 402 is connected to each of the two second internal flow paths 242, and the sensor tester 404 is connected to each of the two fifth internal flow paths 245. In this way, the blood pressure measurement device 1 may have a configuration in which the pump 14 does not include a valve mechanism.

Further, in the example described above, a configuration in which the flow path plate 24a includes the plurality of ports 24c by which the cuffs 52, 54, 62 and the testers 401 to 405 are selectively connected has been described, but the arrangement of the plurality of ports 24c can be set as appropriate. However, collectively disposing the plurality of ports 24c for each of the cuffs 52, 54, 62 to be connected makes it possible to simplify the shapes of the flow path bodies 72, 82, 92 of the cuffs 52, 54, 62, and thus is preferred.

Further, in the example described above, an example is described in which the band 6 is not fixed to the first cuff structure 4 and the second cuff structure 5. However, a configuration may be adopted in which the band 6 is fixed to the second cuff structure 5 which is on the outer side when the first cuff structure 4 and the second cuff structure 5 overlap. That is, even if the band 6 is fixed to the second cuff structure 5, if the first cuff structure 4 closer to the wrist 300 than the second cuff structure 5 is not fixed to the band 6, when the band 6 is tightened, the first cuff structure 4 and the second cuff structure 5 can be tightened and brought into close contact with the wrist 300.

Further, in the example described above, a configuration is adopted in which the device body 3 is provided with the PPG sensor 20a, the SpO2 sensor 20b, and the ECG sensor 20c as the biometric sensors 20 at the bottom portion 31b of the outer case 31 forming the rear surface side of the case 11. However, a configuration may be adopted in which the biometric sensor 20 is any one of the PPG sensor 20a, the SpO2 sensor 20b, and the ECG sensor 20c, or includes a sensor that acquires other biological information in addition to these sensors 20a to 20c or in place of these sensors 20a to 20c.

That is, the present invention is not limited to the above-described embodiments, and various modifications can be made in an implementation stage without departing from the gist thereof. Furthermore, each of the embodiments may be carried out as appropriate in a combination to the extent possible, and combined effects can be obtained in such a case. Furthermore, the inventions at various stages are included in the above-described embodiments, and the various inventions can be extracted in accordance with appropriate combinations in the plurality of disclosed constituent elements. Note that the present invention is not limited to the above-described embodiments, and various modifications can be made in an implementation stage without departing from the gist. Further, embodiments may be carried out as appropriate in a combination, and combined effects can be obtained in such case. Further, the various inventions are included in the embodiment, and the various inventions may be extracted in accordance with combinations selected from the plurality of disclosed constituent elements. For example, in a case in which the problem can be solved and the effects can be obtained even when some constituent elements are removed from the entire constituent elements given in the embodiment, the configuration obtained by removing the constituent elements may be extracted as an invention.

### REFERENCE NUMERALS LIST

1 Blood pressure measurement device
3 Device body
4 First cuff structure
5 Second cuff structure
6 Band
6a Hook-and-loop fastener
6b Knob
11 Case
12 Display unit
13 Operation unit
14 Pump
15 Acceleration sensor
16 Valve
17 Pressure sensor
18 Battery
19 Communication unit
20 Biometric sensor
20a PPG sensor
20b SpO2 sensor
20c ECG sensor
20c1 Electrocardiogra
20d First LED
20e Second LED
20f First PD
20g Second PD
21 Charging circuit unit
22 Memory
23 Processor
24 Flow path plate unit (fluid circuit)
24a Flow path plate
24a1 First port
24a2 Second port
24a3 Third port
24a4 Fourth port
24b Flow path resistor (orifice)
24b1 First flow path resistor
24b2 Second flow path resistor
24cl Fifth port
24c2 Sixth port
24c3 Seventh port
25 Substrate
25a Control board
25b Sensor main board
25c Sensor sub-board
31 Outer case
31a Peripheral wall portion
31b Bottom portion (rear surface)
31b1 Projection portion (sensor unit)
31b1 Sensor unit
31b2 Window portion
31b3 Hole portion
31b4 Opening
31b5 Cover
31b6 Vent
31b7 Waterproof moisture-permeable sheet
31c Loop portion
31d Opening
32 Windshield
33 Cuff cover
33a Opening
33b Hole
41 Button
43 Touch panel
51 First curler
52 First pressing cuff
53 Back plate
54 Sensing cuff
61 Second curler
62 Second pressing cuff
71 Air bag
72 Flow path body
73 Connection portion
81 Air bag
82 Flow path body
83 Connection portion
91 Air bag
92 Flow path body
93 Connection portion
100 Power transmission device
101 Power source
102 Power transmission unit
103 Antenna unit
104 Power transmission terminal
211 Antenna unit
212 Power-receiving unit
213 Charging unit
214 Charging terminal
214a Terminal
241 First internal flow path
242 Second internal flow path
243 Third internal flow path
244 Fourth internal flow path
245 Fifth internal flow path
246 Sixth internal flow path
247 Seventh internal flow path
300 Wrist
311 Radial artery
312 Ulnar artery
401 Pump tester
402 Valve tester
403 First flow path resistor tester
404 Sensor tester
405 Second flow path resistor tester

What is claimed is:

1. A blood pressure measurement device comprising:
a case;
a plurality of components including
a pump provided in the case,
a valve provided in the case, and
a pressure sensor provided in the case; and
a flow path plate unit in which a plurality of internal flow paths are formed, first ends of the plurality of internal flow paths being respectively connected to the plurality of components and second ends of the plurality of internal flow paths being selectively connected to at least one of a plurality of cuffs and a plurality of testers configured to respectively test the plurality of components.

2. The blood pressure measurement device according to claim 1, wherein
the plurality of cuffs include a pressing cuff and a sensing cuff,
the flow path plate unit includes a first flow path resistor and a second flow path resistor, and
the plurality of internal flow paths include
an internal flow path connecting the pressing cuff and the first flow path resistor,
an internal flow path connecting the first flow path resistor and the sensing cuff,
an internal flow path connecting the sensing cuff and the second flow path resistor, and
an internal flow path connecting the second flow path resistor and the atmosphere.

3. The blood pressure measurement device according to claim 2, wherein
the internal flow path connecting the pressing cuff and the first flow path resistor is further connected to the pump, and
the internal flow path connecting the first flow path resistor and the sensing cuff and the internal flow path connecting the sensing cuff and the second flow path resistor are continuously and integrally formed.

4. The blood pressure measurement device according to claim 1, wherein
the flow path plate unit includes a plurality of ports respectively connectable to the plurality of cuffs and the plurality of testers, and respectively continuous with the plurality of internal flow paths.

5. The blood pressure measurement device according to claim 2, wherein
the flow path plate unit includes a plurality of ports respectively connectable to the plurality of cuffs and the plurality of testers, and respectively continuous with the plurality of internal flow paths.

6. The blood pressure measurement device according to claim 3, wherein
the flow path plate unit includes a plurality of ports respectively connectable to the plurality of cuffs and the plurality of testers, and respectively continuous with the plurality of internal flow paths.
